(19) United States
(12) Patent Application Publication
(10) Pub. No.: US 2015/0109307 A1
(43) Pub. Date: Apr. 23, 2015

(54) SYSTEMS AND METHODS TO PRESENT POINTS OF INTEREST IN LONGITUDINAL DATA

(71) Applicant: General Electric Company, Schenectady, NY (US)

(72) Inventor: John Baartz, Tower Lakes, IL (US)

(73) Assignee: General Electric Company, Schenectady, NY (US)

(21) Appl. No.: 14/061,028

(22) Filed: Oct. 23, 2013

(51) Int. Cl. G06T 11/20; A61B 5/044

(52) U.S. Cl. CPC G06T 11/206; A61B 5/044
USPC 345/440.1

(57) ABSTRACT

An example method for displaying longitudinal data includes receiving longitudinal data comprising a plurality of measurements of a variable occurring over a period of time and identifying a first point of interest and a second point of interest in the longitudinal data. The example method includes displaying, in a graph, the first point of interest and the second point of interest in a first time scale. The example method also includes displaying, in the graph, a portion of the longitudinal data occurring between the first point of interest and the second point of interest in a second time scale, which is different than the first time scale.

### RELATED APPLICATIONS

[Not Applicable]

### FEDERALLY SPONSORED RESEARCH OR DEVELOPMENT

[Not Applicable]

### MICROFICHE/COPYRIGHT REFERENCE

[Not Applicable]

### BACKGROUND

Healthcare facilities, such as hospitals or clinics, often employ the use of instruments, equipment and/or other medical devices that generate longitudinal data and/or longitudinal data graphs. Longitudinal data and longitudinal data graphs include one variable measured against another variable (e.g., time) and may be found, for example, in fetal heart monitors, patient electrocardiographs (EKGs), blood pressure monitors, etc. Healthcare practitioners review the longitudinal data graphs to monitor for and identify specific waveforms, patterns and/or features in the data/graph(s) (e.g., a signal) that are indicative of certain events. More specifically, healthcare practitioners are typically concerned with the presence, absence, and/or time duration between certain shapes in the signal. The relationship between these parameters aids in the diagnosis and assessment of the health of a patient.

### BRIEF SUMMARY

Example systems, methods and tangible machine readable storage mediums to present (e.g., display) points of interests in longitudinal data/graph(s) are disclosed herein.

An example method for displaying longitudinal data disclosed herein includes receiving the longitudinal data comprising a plurality of measurements of a variable occurring over a period of time and identifying a first point of interest and a second point of interest in the longitudinal data. The example method includes displaying, in a graph, the first point of interest and the second point of interest in a first time scale. The example method also includes displaying, in the graph, a portion of the longitudinal data occurring between the first point of interest and the second point of interest in a second time scale. In some examples, the second time scale is different than the first time scale.

An example system to display longitudinal data is also disclosed herein that includes an input module to receive longitudinal data, the longitudinal data comprising a variable measured over a period of time. The example system includes a processor to identify a first point of interest and a second point of interest in the longitudinal data and identify a segment of the longitudinal data occurring between the first point of interest and the second point of interest. The example system also includes a dashboard to display a primary graph of the first point of interest in a first time scale, the second point of interest in a second time scale and the segment of the longitudinal data occurring between the first and second points of interest in a third time scale. In some examples, the third time scale is different than the first and second time scales.

Also disclosed herein is a tangible machine readable storage medium comprising instructions that, when executed, cause a machine to at least identify a point of interest in longitudinal data and identify a first section of the longitudinal data occurring prior to the point of interest and a second section of the longitudinal data occurring after the point of interest. The example instructions also cause the machine to display, via a graphical user interface, the longitudinal data in a longitudinal graph, wherein the first section of the longitudinal data is displayed in a first time scale, the second section of the longitudinal data is displayed in a second time scale and the point of interest is displayed in a third time scale, and wherein the third time scale is different than the first time scale and the second time scale.

### BRIEF DESCRIPTION OF SEVERAL VIEWS OF THE DRAWINGS

FIG. 1 illustrates a block diagram of an example healthcare system having an example longitudinal data presentation system.

FIG. 2 shows a block diagram of the example longitudinal data presentation system of FIG. 1.

FIG. 3A shows an example display of a longitudinal graph in a first format (e.g., during automatic identification of data ranges lacking points of interest).

FIG. 3B shows the example display of FIG. 3A with the example longitudinal graph in a second format (e.g., subsequent to reduction in the display size of the data ranges lacking points of interest).

FIG. 3C shows the example display of FIG. 3B with an example secondary graph to indicate the amount of time compression (e.g., time scale adjustment) occurring in the example longitudinal graph.

FIG. 3D shows the example display of FIG. 3B with an alternative secondary graph to indicate the amount of longitudinal data that has been compressed in the example longitudinal graph.

FIG. 4A shows an example display of a longitudinal graph in a first format (e.g., during manual identification of data ranges lacking points of interest).

FIG. 4B shows the example display of FIG. 4A with the example longitudinal graph in a second format (e.g., subsequent to reduction in the display size of the data ranges lacking points of interest) and an example secondary graph.

FIG. 5 is a flow diagram illustrating an example method of reducing the display size of ranges of data/graph(s) in an example longitudinal graph.

FIG. 6 shows a block diagram of an example processor system that may be used to implement systems and methods described herein.

The foregoing summary, as well as the following detailed description of certain examples of the present invention, will be better understood when read in conjunction with the appended drawings. For the purpose of illustrating the invention, certain examples are shown in the drawings. It should be understood, however, that the present invention is not limited to the arrangements and instrumentality shown in the attached drawings.

### DETAILED DESCRIPTION OF CERTAIN EMBODIMENTS

Although this specification discloses example methods, systems and machine readable medium including, among other components, software and/or firmware executed on hardware, it should be noted that such systems are merely illustrative and should not be considered as limiting. For example, it is contemplated that any or all of these hardware, software, and firmware components could be embodied exclusively in hardware, exclusively in software, or in any combination of hardware and software. Accordingly, while the following describes example methods, systems, and machine readable mediums, persons of ordinary skill in the art will readily appreciate that the examples provided are not the only way to implement such methods, systems and machine readable medium.

The present disclosure relates to identifying and presenting points of interest in longitudinal graphs. More specifically, the present invention relates to systems and methods to more effectively present points of interest in longitudinal graphs by reducing the display size of ranges of data falling outside of or between the points of interest, which enables more points of interest to be presented in a viewable display area.

Longitudinal data includes a first variable measured against a second variable. Often, the second variable is time, and the first variable is an input from a sensor or other device. Many healthcare facilities utilize instruments that display longitudinal data in the form of longitudinal data graphs (e.g., plots, charts, etc.). For example, a patient's heart rate can be measured using an electrocardiograph (EKG or ECG) that graphs the electrical activity of the heart over a period of time. In another example, fetal heart monitoring is used to track how well a baby is doing within a contracting uterus and for detecting signs of distress. External transducers are connected to a mother's abdomen and information is sent to a fetal heart monitor, which records the fetal heart rate on a continuous strip of paper. The strip of paper includes the fetal heart rate plotted over a span of time.

Longitudinal graphs generated by this data can grow to be extremely large due to the length of time the variable is recorded. Healthcare practitioners (e.g., nurses, doctors, physicians, etc.) review these graphs to monitor for specific point(s) of interest (POIs) (e.g., waveforms, patterns, features, signal shapes, dips, spikes, etc.), the absence of POIs and/or the duration between POIs. POIs and their relationship to one another are indicative of certain events or issues occurring with the patient. In some instances, a patient may be monitored for a relatively long period of time and the respective longitudinal data graph becomes extensive. To increase workflow, healthcare practitioners need to review the longitudinal data graphs as quickly and as accurately as possible. Currently, healthcare practitioners scroll through these large graphs, either on a digital monitor or manually through a strip of graph paper, to review the data and make an assessment. However, in some examples, the longitudinal graphs may include data recorded over days or weeks of time. With such a lengthy graph, this process can become quite complicated and time consuming Whether the graph is printed on paper or displayed on a monitor, the longitudinal graph may include ranges (e.g., areas, sections, segments, portions, spans, lengths) of data that are void of POIs (e.g., uninteresting time spans) and which the healthcare practitioner scrolls through in order to approach the next POI. In some examples, the ranges of data lacking POIs are unimportant to the practitioner and, thus, the practitioner spends times scrolling past these areas in the longitudinal data graphs.

For example, a cardiologist may want to view an EKG on a digital screen/monitor and the EKG may have been recorded over a weeklong period. Because of the size of the monitor (e.g., the display area, the screen real estate), only a portion (e.g., a few minutes) of the EKG is viewable on the monitor at one time. For example, if the monitor is ten (10) inches (in) across, and the time scale of the EKG is presented as 10 s/inch, only 100 s of data is viewable on the monitor at one time. Therefore, the cardiologist scrolls (e.g., via a scroll bar) through the EKG while looking for certain POIs in the data/graph. However, in some examples, the EKG includes only a few POIs and the rest of the EKG lacks POIs (e.g., is unimportant to the cardiologist) and, thus, the cardiologist spends significant time scrolling through these portions of the EKG lacking POIs.

The example methods and systems for displaying longitudinal data graphs described herein advantageously identify POIs and/or ranges of data lacking POIs and reduce the display size of the ranges of data lacking POIs to more effectively display the POIs in a longitudinal data graph. By compressing or decreasing the length of the ranges of data lacking POIs, the POIs are presented closer together (e.g., a visual co-location) and, thus, more POIs can be presented in the relatively same viewable display window (e.g., a full fidelity). As a result of optimizing clinical recognition of data, a healthcare practitioner can more quickly identify and assess the POIs and make an appropriate diagnosis. Also, because the ranges of data lacking the POIs are still presented (rather than deleted or omitted), the overall trends in these ranges are still viewable in the longitudinal data graphs. Thus, more POIs are presented in the same viewable display area while the data occurring in the reduced ranges is still preserved and viewable. In some examples, the trends over the ranges of data lacking POIs are important but difficult to realize with a larger graph. By displaying the data in the reduced ranges these trends are more easily recognized. The methods and systems more effectively utilize the display screen real estate to present the longitudinal data/graph(s).

In some examples, the presentation methods and systems described herein provide a secondary graph (e.g., a compression graph) to illustrate the amount of compression (e.g., the change in time scale) occurring in the primary graph (e.g., the main longitudinal graph). The secondary graph informs a viewer of the areas in the primary graph that have been altered (e.g., where the time scale has been increased or decreased to reduce the display size), at what rate and/or how much these areas have been altered. Thus, a viewer can visually see the amount of time compression (e.g., dilation, alteration) occurring through the different ranges of data.

Although the methods, systems and machine readable storage mediums disclosed here are described in regards to healthcare applications, it is to be understood that the present methods, systems and machine readable storage mediums can also be used to present longitudinal data graphs in any other industry/application. For example, the disclosed systems and methods can also be used in financial markets for viewing stock prices. For instance, a stockbroker may desire to view only the peaks (e.g., POIs) in the price of a stock that exceed a certain dollar value. The example longitudinal data presentation system reduces the display size of ranges in time of unimportant data occurring between the desired peaks and presents the peaks closer together so that the broker may view more of the peaks in the same viewable display window.

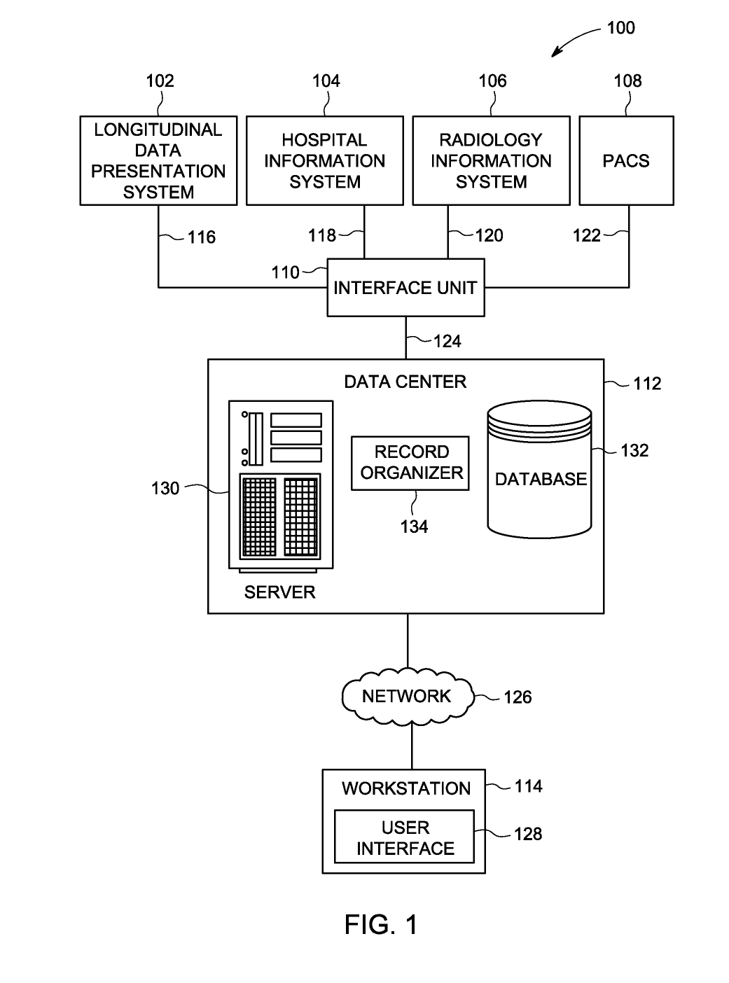

Turning now the figures, FIG. 1 shows a block diagram of an example healthcare system 100 having an example longitudinal data presentation system 102, which, in some examples, identifies POIs in a longitudinal data graph and/or ranges of data lacking POIs and reduces the display size of the ranges of data lacking POIs to display more POIs and more of the longitudinal graph in the viewable display area, as described in further detail below. The example healthcare system 100 includes the longitudinal data presentation system 102, a hospital information system (HIS) 104, a radiology information system (RIS) 106, a picture archiving and communication system (PACS) 108, an interface unit 110, a data center 112, and a workstation 114. In the illustrated example, the longitudinal data presentation system 102, the HIS 104, the RIS 106, and the PACS 108 are housed in a healthcare facility and/or locally archived. However, in other implementations, the example longitudinal data presentation system 102, the HIS 104, the RIS 106, and/or the PACS 108 can be housed one or more other suitable locations. In certain implementations, one or more of the longitudinal data presentation system 102, the HIS 104, the RIS 106, the PACS 108, . . . etc., can be implemented remotely via a thin client and/or downloadable software solution. Furthermore, one or more components of the healthcare system 100 can be combined and/or implemented together. For example, the longitudinal data presentation system 102, the RIS 106 and/or the PACS 108 can be integrated with the HIS 104; the PACS 108 can be integrated with the RIS 106; and/or the longitudinal data presentation system 102 and/or the three example information systems 104, 106, and/or 108 can be integrated together. In other example implementations, the healthcare system 100 includes a subset of the longitudinal data presentation system 102 and/or the illustrated information systems 104, 106, and/or 108. For example, the healthcare system 100 may include only one or two of the HIS 104, the RIS 106, and/or the PACS 108. Information (e.g., scheduling, test results, observations, diagnosis, etc.) can be entered into the HIS 104, the RIS 106, and/or the PACS 108 by healthcare practitioners (e.g., radiologists, physicians, and/or technicians) before and/or after patient examination.

The example longitudinal data presentation system 102 identifies POIs in longitudinal data/graph(s) and/or ranges of data lacking POIs and reduces (e.g., compresses, alters, changes) the display size of the ranges of data lacking POIs to more efficiently present (e.g., display) the respective POIs in the longitudinal graph. The longitudinal data presentation system 102 may be connected to any instrument (e.g., equipment, medical device, sensor, etc.) to receive, record and/or store the longitudinal data. In some examples, the longitudinal data presentation system 102 includes a user interface and/or workstation for presenting and/or interacting with the longitudinal data/graph(s). In other examples, the longitudinal data presentation system 102 may be accessed by the workstation 114, described in detail below. In some examples, the longitudinal data presentation system 102 is combined and/or implemented with one or more of the other components of the system 100 (e.g., the longitudinal data presentation system 102 is combined with the workstation 114). Additionally or alternatively, the longitudinal data presentation system 102 may receive longitudinal data/graph(s) from one or more of the other components of the system 100 such as, for example, from the information systems 104, 106, 108 that store and maintain patient medical data and reports.

The HIS 104 stores medical information such as clinical reports, patient information, and/or administrative information received from, for example, personnel at a hospital, clinic, and/or a physician's office. The RIS 106 stores information such as, for example, radiology reports, messages, warnings, alerts, patient scheduling information, patient demographic data, patient tracking information, and/or physician and patient status monitors. Additionally, the RIS 106 enables exam order entry (e.g., ordering an x-ray of a patient) and image and film tracking (e.g., tracking identities of one or more people that have checked out a film). In some examples, information in the RIS 106 is formatted according to the HL-7 (Health Level Seven) clinical communication protocol.

The PACS 108 stores medical images and data (e.g., x-rays, scans, three-dimensional renderings, digital version of fetal heart monitor strips, etc.) such as, for example, digital images in a database or registry. In some examples, the medical images and data are stored in the PACS 108 using the Digital Imaging and Communications in Medicine (“DICOM”) format. Images and data are stored in the PACS 108 by healthcare practitioners (e.g., imaging technicians, cardiologists, physicians, radiologists) after a medical imaging of a patient and/or are automatically transmitted from medical imaging devices to the PACS 108 for storage. In some examples, the PACS 108 can also include a display device and/or viewing workstation to enable a healthcare practitioner or provider to communicate with the PACS 108. As mentioned above, one or more of the HIS 104, the RIS 106 and/or the PACS 106 may include reports or data including longitudinal data/graphs, which can be transferred to and/or accessed by the longitudinal data presentation system 102.

In other examples, additional information systems may be integrated into the system 100 such as, for example, clinical information systems (CIS), cardiovascular information systems (CVIS), and additional storage systems such as library information systems (LIS) and electronic medical records (EMR), which may also be connected to the interface unit 110. In some examples, one or more of these systems include longitudinal data/graphs that can be transferred to and/or accessed by the longitudinal data presentation system 102.

The interface unit 110 includes a longitudinal data presentation system connection 116, a hospital information system interface connection 118, a radiology information system interface connection 120, a PACS interface connection 122, and a data center interface connection 124. The interface unit 110 facilitates communication among the longitudinal data presentation system 102, the HIS 104, the RIS 106, the PACS 108, and/or the data center 112. The interface connections 116, 118, 120, 122 and 124 can be implemented by, for example, a Wide Area Network (“WAN”) such as a private network or the Internet. Accordingly, the interface unit 110 includes one or more communication components such as, for example, an Ethernet device, an asynchronous transfer mode (“ATM”) device, an 802.11 device, a DSL modem, a cable modem, a cellular modem, etc. In turn, the data center 112 communicates with the workstation 114, via a network 126, implemented at a plurality of locations (e.g., a hospital, clinic, doctor's office, other medical office, or terminal, etc.). The network 126 is implemented by, for example, the Internet, an intranet, a private network, a wired or wireless Local Area Network, and/or a wired or wireless Wide Area Network. In some examples, the interface unit 110 also includes a broker (e.g., a Mitra Imaging's PACS Broker) to allow medical information and medical images to be transmitted together and stored together.

The interface unit 110 receives longitudinal data/graph(s), images, medical reports, administrative information, and/or other clinical information from the longitudinal data presentation system 102 and/or the information systems 104, 106, 108 via the respective interface connections 116, 118, 120, 122. If necessary (e.g., when different formats of the received information are incompatible), the interface unit 110 translates or reformats (e.g., into Structured Query Language (“SQL”) or standard text) the medical information (e.g., patient identification data, longitudinal data/graph(s), medical reports, etc.) to be properly stored at the data center 112. The reformatted medical information can be transmitted using a transmission protocol to enable different medical information to share common identification elements, such as a patient name or social security number. Next, the interface unit 110 transmits the medical information to the data center 112 via the data center interface connection 124. Finally, medical information is stored in the data center 112 in, for example, the DICOM format, which enables medical images and other corresponding medical information to be transmitted and stored together.

The medical information is later viewable and easily retrievable at the workstation 114 (e.g., by their common identification element, such as a patient name or record number). The workstation 114 can be any equipment (e.g., a personal computer) capable of executing software that permits electronic data (e.g., medical reports) and/or electronic medical images (e.g., x-rays, ultrasounds, MRI scans, etc.) to be acquired, stored, or transmitted for viewing and operation. The workstation 114 receives commands and/or other input from a user via, for example, a keyboard, mouse, track ball, microphone, etc. The workstation 114 is capable of implementing a user interface 128 to enable a healthcare practitioner to interact with the healthcare system 100.

The example data center 112 of FIG. 1 is an archive to store information such as, for example, images, data (e.g., in the form of longitudinal data/graph(s)), medical reports, and/or, more generally, patient medical records. In addition, the data center 112 can also serve as a central conduit to information located at other sources such as, for example, data generators (e.g., the longitudinal data presentation system 102), local archives, hospital information systems/radiology information systems (e.g., the HIS 104 and/or the RIS 106), or medical imaging/storage systems (e.g., the PACS 108 and/or connected imaging modalities). That is, the data center 112 can store links or indicators (e.g., identification numbers, patient names, or record numbers) to information. In the illustrated example, the data center 112 is managed by an application server provider (“ASP”) and is located in a centralized location that can be accessed by a plurality of systems and facilities (e.g., hospitals, clinics, doctor's offices, other medical offices, and/or terminals). In some examples, the data center 112 can be spatially distant from the longitudinal data generator 102, the HIS 104, the RIS 106, and/or the PACS 108 (e.g., at General Electric® headquarters).

The example data center 112 of FIG. 1 includes a server 130, a database 132, and a record organizer 134. The server 130 receives, processes, and conveys information to and from the components of the healthcare system 100. The database 132 stores the medical information described herein and provides access thereto. The example record organizer 134 of FIG. 1 manages patient medical histories, for example. The record organizer 134 can also assist in procedure scheduling, for example.

In certain examples, the longitudinal data presentation system 102 is located in the PACS 108. In an alternative example, the longitudinal data presentation system 102 may be located separately or may be included in any other component of the healthcare system 100. In some examples, the longitudinal data presentation system 102 is connected directly to the workstation 114 and user interface 128. In some examples, the workstation 114 and user interface 128 are incorporated directly into the longitudinal data presentation system 102. For example, a fetal heart rate monitor may include a user interface (e.g., the user interface 128) and workstation (e.g., the workstation 114) to monitor and display the fetal heart rate in real time.

The longitudinal data presentation system 102 alters the presentation of longitudinal data/graph(s) to more effectively display the POIs (e.g., important waveforms, patterns, spikes, peaks, dips, etc.). As mentioned above, over a large period of time, longitudinal graphs may become relatively lengthy. Users may only be concerned with certain features (e.g., POIs) in the data/graph(s). In some examples, there may be relatively long lengths of time between these features. Therefore, not all of the longitudinal graph can fit within the viewable display window and a user has to scroll through all of the graph to search for these features, which may require significant time if there are long ranges of data lacking these features. The example longitudinal data presentation system 102 identifies POIs and/or ranges of data lacking POIs (e.g., ranges that are candidates for compression) and reduces the display size of the ranges of data lacking POIs to display the POIs closer together.

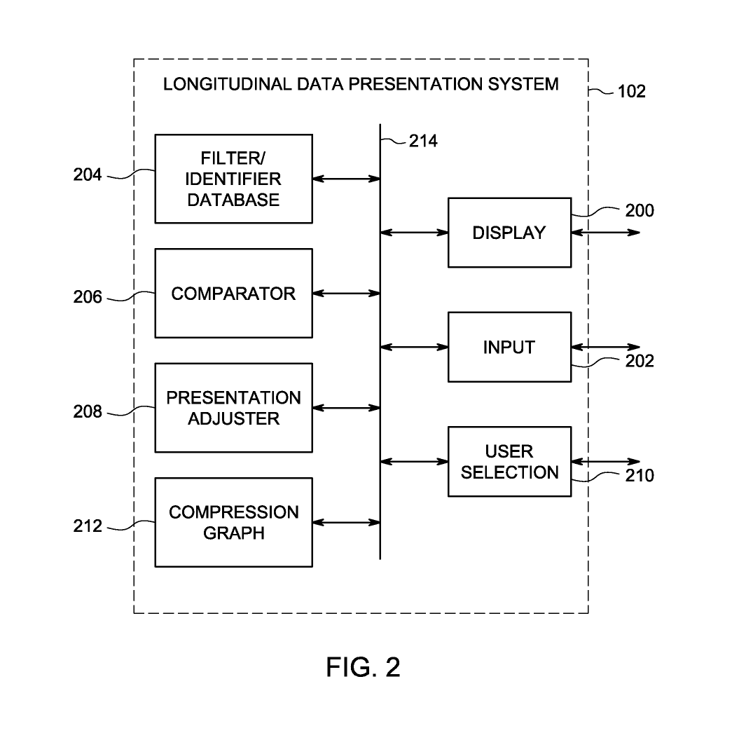

FIG. 2 is a block diagram of the example longitudinal data presentation system 102. The example presentation system 102 includes a display module 200, which may be, for example, a module that interacts with the workstation 114 and/or user interface 128 from the system 100 shown in FIG. 1. In other examples, the display module 200 may connect to any computer screen, image viewer and/or other display device known to those skilled in the art that is capable of presenting (e.g., displaying) longitudinal graph(s). In some examples, the display module determines the amount display space available (e.g., viewable display area, screen real estate) and/or resolution of the longitudinal data/graph to be displayed on the screen. The available display space and the resolution may be used in determining how much space is available (e.g., in one or more monitors showing one or more items) given a manually and/or automatically selected range of data/graph to compress, as described in further detail below.

In the example shown, the presentation system 102 also includes an input module 202 for receiving longitudinal data/graph(s). The input module 202 may receive data directly from an instrument used to gather the longitudinal data (e.g., an EKG, an EEG, a blood pressure monitor, a sensor, a transducer, etc.) and/or from one or more other systems connected to the presentation system (e.g., the HIS 104, the RIS 106, the PAC 108).

As shown, the example presentation system 102 also includes a filter/identifier database 204. Depending on the industry or application, certain waveforms, features and/or patterns, e.g., POIs, in the longitudinal data are considered important. These POIs are typically known and the user generally looks for these POIs as an indication of a certain event. In some examples, the filter/identifier database 204 stores one or more predetermined waveforms or patterns. The filters/identifiers may include any parameters that define the shape or profile of the respective filter/identifier. In other examples, the filter/identifier database 204 may include profiles used to identify ranges of data occurring between points of interest. For example, the filter/identifier database 204 may include instructions to seek out a profile in the graph that is relatively flat or even (e.g., absent of POIs, uninteresting, absent of data, normal data, steady).

The example presentation system 102 also includes a comparator 206, which compares a selected filter/identifier with the longitudinal data/graph. As mentioned above, a user may only want to view POIs, which may include, for example, a certain shape in the graph (e.g., a spike occurring at a first rate, a peak over a first amount, a plateau, etc.). After choosing the appropriate filter/identifier, the comparator 206 analyzes the longitudinal data/graph for sections of the data/graph that substantially correspond or match the selected filter/identifier. As mentioned above, each of the filters/identifiers may include a list of parameters that define the boundaries of the respective POI. The system analyzes the longitudinal data/graphs for sections of data matching one or more of these parameters to make a determination if a section should be considered a POI. In other examples, as mentioned above, the selected filter/identifier identifies the ranges of data lacking POIs (e.g., ranges of data that are quiet or uninteresting).

The example presentation system 102 also includes a presentation adjuster 208. As described above, the comparator 206 can identify the POIs and/or the ranges of data lacking POIs in the longitudinal data/graph based a selected filter/identifier. After identifying the POIs (e.g., the features or ranges of data that are important) and/or ranges of data lacking POIs, the presentation adjuster 208 adjusts the presentation of the longitudinal graph. The presentation adjuster 208 identifies a range of data occurring between two identified POIs and increases the time scale occurring during the respective range, so that when the longitudinal graph is presented, the area of the display occupied by the range of data is relatively narrower and, thus, the POIs on either side of the range of data appear closer together. The presentation system 102 may perform this function one or more times depending on the length of the longitudinal graph and the amount of ranges of data lacking POIs.

In other examples, a user may manually choose which ranges of data to present (e.g., POIs) or preserve in the viewable display area and/or which ranges of data to reduce (e.g., ranges of data that are unimportant or lack points of interest) in the viewable display area. The presentation system 102 includes a user selection module 210 that receives instructions (e.g., a selected range) from the user and communicates with the presentation adjuster 208 to adjust the presentation of the longitudinal data/graph accordingly. For example, a user may view the graph and determine there is a large area of data that is unimportant. The user may highlight (e.g., via a mouse) a section of the graph to compress. The user selection module 210 receives these instructions from the user and communicates with the presentation adjuster 208 to reduce the display area occupied by the section and selected by the user.

In some examples, the amount of compression is based on the amount of available screen space (e.g., determined by the display module 200) and/or the amount of data/graph(s) to be displayed. For example, if only a few POIs are identified, the presentation system 102 determines the amount of compression required so that all of the POIs are viewable in the same display area. The system 102 uses the available display space and resolution parameters in determining the amount of compression to occur between the POIs to fit the POIs in the viewable display area.

In the example shown, the presentation system 102 also includes a compression graph module 212. The compression graph module 212 determines the amount of compression (e.g., change in time scale) occurring in the compressed areas and provides a compression graph (e.g., a secondary graph, a secondary presentation) to indicate the amount of time compression occurring. For example, a compression graph (e.g., a line map) may be displayed directly above the longitudinal data/graph. The X-axis of the compression graph aligns with the relative location in the longitudinal graph and the Y-axis indicates the amount of compression occurring at the point along the X-axis. If there is a compressed area in the longitudinal graph, the compression graph increases to indicate the time scale occurring in that area has increased and, thus, the data/graph has been compressed. In some examples, the Y-axis indicates the time scale occurring in the longitudinal data/graph.

In other examples, the longitudinal graph may be represented as a three-dimensional (3D) graph and the sections of the graph between the points of interest are bent or curved into the screen to visually move the points of interest closer together. In such an example, the compression graph or secondary graph displays a line representing the shape of the top edge of the graph and, thus, a depth and/or amount of compression occurring across the compressed sections can be visualized.

In the example shown, the display module 200, the input module 202, the filter/identifier database 204, the comparator 206, the presentation adjuster 208, the user selection module 210 and/or the compression graph module 212 are in communication with each other via a bus 214. However, in other examples, the display module 200, the input module 202, the filter/identifier database 204, the comparator 206, the presentation adjuster 208, the user selection module 210 and/or the compression graph module 212 may be located offsite or embodiment in another device such as, for example, one or more of the components of the healthcare system 100 shown and described in FIG. 1.

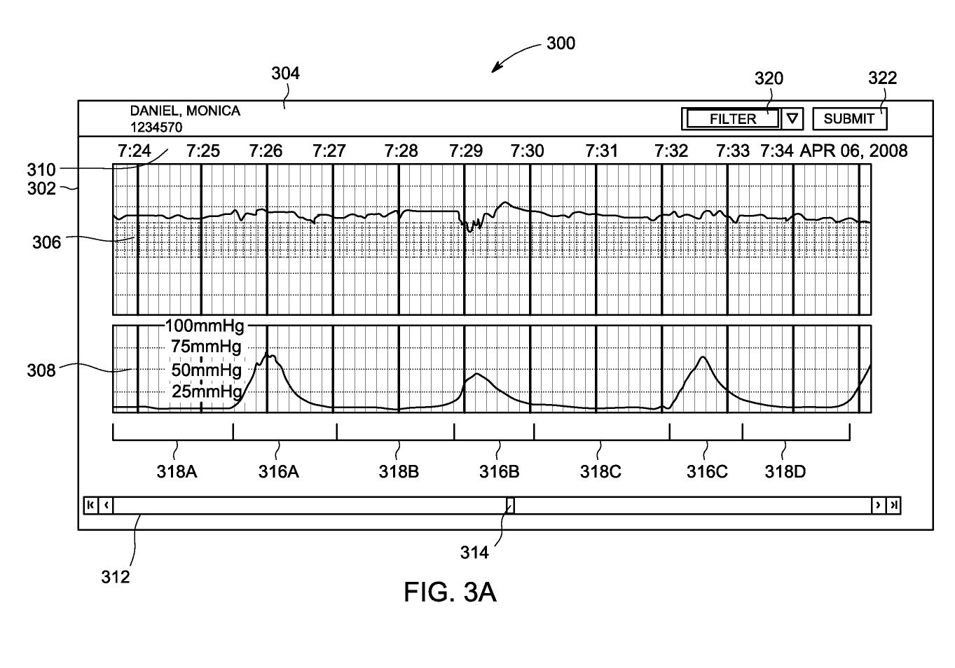

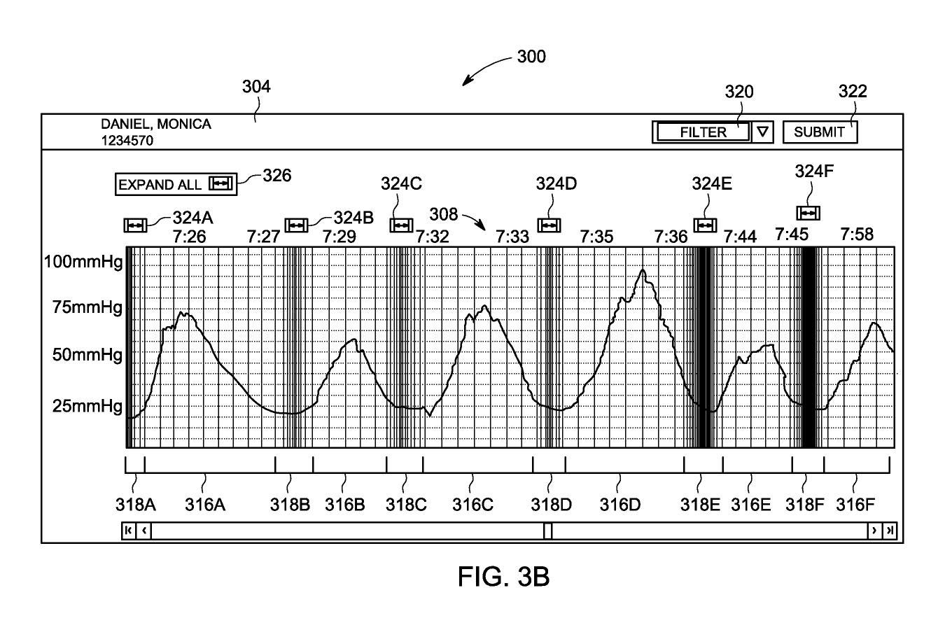

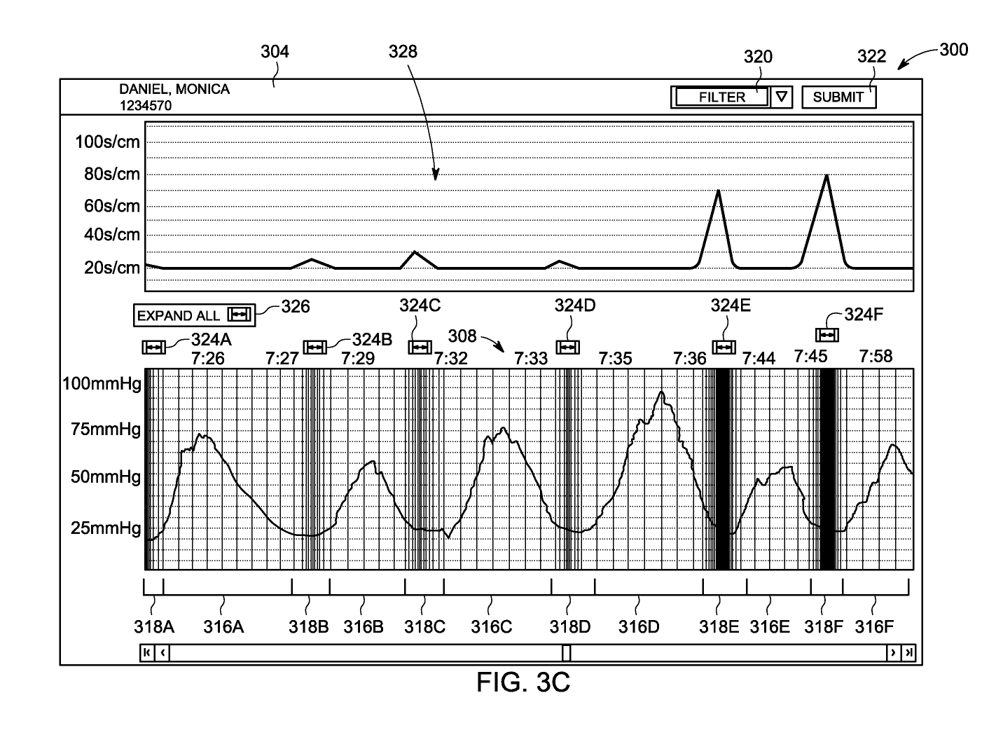

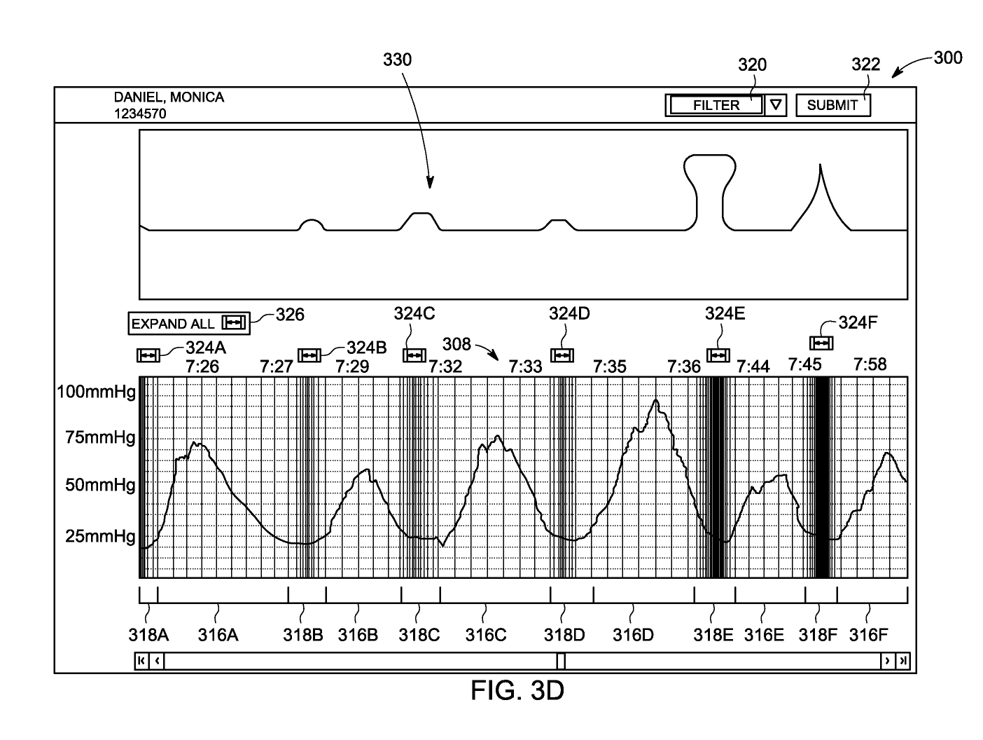

FIG. 3A shows an example screen 300 (e.g., a user interface, a monitor, a presentation display, a dashboard) having an application window 302. In the example shown, the application window 302 includes two longitudinal graphs. Specifically, the application window 302 is displaying an example of a fetal heart rate signal and corresponding contractions of the mother. The application window 302 includes a header portion 304 that identifies the application window and/or other identifying information of the file or patient being viewed. As shown, the upper graph 306 is the fetal heart rate (in beats per minute (bpm)) and the lower graph 308 is the mother's contractions (in millimeters (mm) of mercury (Hg)).

In the example application window 302 shown, the top of the upper graph 306 includes a row of time stamps 310. In this example, each bold vertical line in the graphs 308, 310 represents one minute and the time of the data is displayed above the respective line. The lighter vertical intermediate lines between the bold vertical lines represent ten (10) second segments (e.g., ranges, increments, etc.). Therefore, there are six columns (or squares) between each one minute time stamp. In the example shown, two squares or columns (e.g., twenty (20) seconds) are about one centimeter (cm) in length on the screen 300. Therefore, each minute of the graphs 306, 308 on the screen 300 is about three centimeters (3 cm) in length.

The lower graph 308 represents the mother's contractions. Closer to the time of birth, it is important to monitor a mother's contractions for repeating and increasing occurrences or certain waveforms in the longitudinal graph. As mentioned above, if each minute of the graph is about three centimeters (3 cm) in length, the entire viewable display area of the application window 302 is about four and a half inches (4.5) long and display about eleven and a half (11.5) minutes of data. In some examples, a mother's contractions may be monitored for hours, days or even weeks. Therefore, the graph 308 can grow and become relatively lengthy over this period of time. When viewing the graph, healthcare practitioners are concerned with the occurrences of the contractions (e.g., POIs) and/or the time between the contractions. However, with such a long graph, making a determination can become complicated and require relatively large amounts of time because only a portion of the graph (e.g., about eleven and a half (11.5) minutes) is displayed in the viewable display area at one time.

The bottom of the application window 302 includes a scrollbar having a trough 312 (e.g., a track) and a bar 314 (e.g., a thumb). In order to view earlier or later parts of the graph, a user can click and drag the bar 314 along the trough 312 to present other portions of the graph not visible on the screen. With such a long graph, viewing this data and making an assessment becomes difficult because there may be a large amount of contractions and/or periods between contractions.

As shown in the portion of the graph 308 visible in the application window 302 in FIG. 3A, there are three contractions 316A, 316B, 316C (e.g., POIs) and four ranges (e.g., sections, portions, spans, segments, etc.) in time where there are no contractions, non-contraction areas 318A, 318B, 318C, 318D (e.g., uninteresting spans of the data/graph). The non-contraction areas 318A-D occupy a relatively large area of the screen space. Thus, a practitioner may be required to scroll through the remaining graph (e.g., portions of the graph not presently visible on the screen) to skip past the non-contraction areas to get a full understanding of the mother's contractions.

In some examples, the presentation system 102 (FIG. 2) includes a plurality of filters/identifiers that can be selected and used to filter the longitudinal data/graph to identify the POIs. In the example shown in FIG. 3A, the application window 302 includes a filter dropdown menu 320. The filter dropdown menu includes a list of potential filters or predetermined waveform patterns that a user may choose from. In other examples, a separate screen or menu may be accessed for selecting a filter/identifier. The filters included in the dropdown menu 320 may be industry/application specific. For example, the types of waveforms patterns or signal shapes that are important in viewing fetal heart rates and contractions may include a one set of waveform patterns and the waveform patterns or signal shapes important when viewing stock prices may include another set of waveform patterns or signal shapes. After a user selects a filter from the dropdown menu 320, the user may press a submit button 322 to apply the filter. The selected filter or waveform pattern is compared to the longitudinal data/graph and ranges of data/graph in the longitudinal data/graph that substantially match the filter or waveform pattern are identified (e.g., as POIs). As described above, this operation may be performed by the comparator 206 of the presentation system 102 shown in FIG. 2. Once the POIs are identified, the portions of the data/graph occurring between the POIs (e.g., the ranges lacking points of interest, unimportant data ranges) are identified and may be compressed (e.g., reduced display size) to bring the POIs closer together in the application window 302 (e.g., the viewable display area).

The presentation system 102, as described above and shown in FIGS. 1 and 2, identifies the POIs in the longitudinal data/graph(s) and/or areas that are absent of POIs and compresses the areas that are absent of POIs to display more POIs in the viewable screen.

FIG. 3B illustrates the lower graph 308 after the non-contraction areas 318A, 318B, 318C, 318D (e.g., the ranges of data lacking POIs, the unimportant ranges of data) have been compressed (e.g., reduced in display size). The reference numbers used in FIG. 3A have been reproduced in FIG. 3B to indicate the same or similar elements. In example screen 300 shown in FIG. 3B, only the lower graph 308 is reproduced. However, in other examples, the upper graph 306 can likewise be adjusted according the teachings of this disclosure and presented above the lower graph 308.

As shown, the original three contractions 316A, 316B, 316C and three additional contractions 316D, 316E, 316F are now viewable in the application window 302 (e.g., the viewable display area) of the screen 300. The area occupied by the non-contraction areas 318A, 318B, 318C, 318D has been reduced, as well as additional non-contraction areas 318E, 318F, which are also now viewable in the application window 302 of the screen 300. In this example, the non-contraction areas 318A-F were identified as ranges of data lacking POIs (e.g., candidates for compression). Thus, more contractions 316A-F (e.g., POIs) can be viewed in the application window 302, while still presenting all of the data/graph (e.g., rather than omitting or deleting certain sections of the data/graph). Specifically, the presentation system displays the non-contraction areas 318A-F in a time scale or rate that is higher (e.g., above the normal time scale of 20 s/cm) than the contractions 316A-F on either side of the respective non-contraction areas 318A-F. A higher time scale results in reduction of the horizontal length of the columns or segments on the screen 300. In the example shown, the original time scale is one minute for every 3 cms, but a higher time scale may be represented by a few minutes over the same distance. Thus, the data within the higher time scales areas is compressed and takes up less space in the viewable display area.

As shown in the example of FIG. 3B, the first contraction 316A takes up the same amount of screen space as does the first contraction 316A shown in FIG. 3A. However, the non-contraction area 318B occurring just after the first contraction 316A has been reduced in width on the screen as shown in FIG. 3B. As shown, the vertical time lines are moved closer together because the time scale has been increased and, thus, more time passes during the non-contraction area 318B along the X-axis. In this example, each square or column still represents ten seconds (10 s) of time, but the squares or columns have been moved closer together, so that a great amount of time is covered in less display area. Because of this compression, the second contraction 316B is moved closer to the first contraction 316A in the application window 302. As shown, the first contraction area 316A is graphed (e.g., displayed) in a first time scale (e.g., 1 minute/3 centimeters (cm)), the second contraction area 316B is graphed in a second time scale (e.g., 1 minute/3 cm), which may or may not be the same as the first time scale, and the non-contraction area 318B is graphed in a third time scale. In the example shown, the first and second time scales are the same and the third time scale is higher than the first and second time scales. In other examples, the first and second time scales may also be different than each other.

The non-contraction areas 318A-F may be compressed in different manners. For example, as shown in FIG. 3B, the time scale of the non-contraction areas 318A-F increases and decreases (e.g., linearly, exponentially) throughout the respective non-contraction areas. As shown, the first half of each of the respective non-contraction areas 318A-F increases in time scale until the middle point of each of the respective non-contraction areas 318A-F, and the second half of each of the respective non-contractions areas 318A-F decreases in time scale until the end of each of the respective non-contraction areas 318A-F, at which point the time scale aligns with the respective contraction area 316A-F immediately after. However, in other examples, the compressed areas may be presented in a linear or constant time scale (e.g., double the original time scale, 40 s/cm). In some examples, the amount of compression is based on the amount of available screen space (e.g., determined by the display module 200 in FIG. 2) and/or resolution of the longitudinal data/graph(s). For example, if only a few POIs are identified, the ranges of data lacking POIs may be compressed enough to fit all of the POIs in the display space of the application window 302 on the screen 300. In some examples, the compressed version of the graph 308 may be printed out.

Also shown in FIG. 3B are expand buttons 324A, 324B, 324C, 324D, 324E. As shown, each of the expand buttons 324A-F is displayed above a respective compressed area 318A-F and corresponds (e.g., is associated with) to that respective area. The expand buttons 324A-F indicate an area in the data/graph has been compressed and the user may select (e.g., click) one or more of the buttons 324A-F to expand a respective compressed area. In the example shown, the expand buttons 324A-F are represented as icons having a rectangle with two outward facing arrows. In other examples, the expand buttons 324A-F may be represented by any logo, symbol, letter, indicia, icon, number, etc. Additionally, an expand all button 326 is also provide to expand all of the compressed areas (e.g., the non-contraction areas 318A-F).

In some examples, such as the example shown in FIG. 3C, a secondary graph 328 (e.g., a secondary presentation, a compression graph) is displayed to illustrate the amount of compression (e.g., time scale adjustment) occurring in the primary graph (e.g., the main longitudinal graph, the contraction graph 308, the primary presentation). The reference numbers used in FIGS. 3A and 3B have been reproduced in FIG. 3C to indicate the same or similar elements. The X-axis of secondary graph 328 aligns with the location in the contraction graph 308 below the secondary graph 328 and the Y-axis of the secondary graph 328 indicates the amount of compression (e.g., the change in time scale, the depth of compressed time). As shown, the points in the secondary graph 328 above the non-contraction areas 318A-F (e.g., the sections of the secondary graph that align with the non-contraction areas 318A-F) spike in time scale and, thus, represent an increase in time scale occurring in the graph 308. In other words, the amount of time occurring in the graph 308 during the non-contraction areas 318A-F over the relatively same distance is greater (e.g., more time elapses per unit of distance). The secondary graph 328 provides a visual indication of the amount of compression (e.g., time scale adjustment, alteration, distortion, change) occurring in the primary graph 308.

As shown in FIGS. 3B and 3C, the time scale of non-contraction areas 318A-F is higher than the contractions 316A-F and, thus, the contractions 316A-F occurring on either side of the respective non-contraction areas 318A-F are displayed closer together (e.g., visual co-location). The non-contraction areas 318E and 318F cover a relatively longer period of time. As shown in the secondary graph 324, the time scale spikes occurring during the non-contraction areas 318E and 318F are higher than the spikes occurring during the non-contractions areas 318B-D. The vertical time lines in the contraction graph 308 are so close together that a solid appearing line is shown. In some examples, this shading is an indication of the amount compression. In the example shown, the data occurring during these periods may become obstructed because the vertical time lines have been moved close together. However, in other examples, the vertical time lines may be a lighter shade of color such that the data occurring during these periods can still be seen.

In the example shown in FIG. 3C, the secondary graph 328 is located above the contraction graph 308. However, in other examples, the second graph 328 may be located below, adjacent and/or separate (e.g., on another screen, in another page of the application window 302) from the contraction graph 308. The secondary graph 328 may be provided by, for example, the compression graph module 212 of the presentation system 102 shown in FIG. 2. The compression graph module 212 determines the amount of compression occurring in primary graph and presents this compression as the secondary graph to indicate the amount of compression occurring in the primary graph.

In some examples, the compressed areas 318A-F of the graph 308 shown in FIGS. 3B and 3C can be thought of as bending or curving backwards or away from the user, e.g., similar to folds in a curtain or ribbon. The visual effect of bending these compressed areas backwards brings the POIs closer together, and the area (e.g., horizontal distance) between the POIs is reduced in width into the curvature of these bends (e.g., a curtain effect, a ribbon effect). The data in the area occurring in the curve (e.g., the recession, the bend, etc.) is still viewable but the data occurs over a shorter horizontal distance and the vertical time lines appear closer together because each square still represents a ten (10) second time increment. Thus, all of the data of the longitudinal graph is still viewable, and the POIs are brought closer together by reducing the amount of horizontal screen space these areas take up. Additionally, in some examples, the secondary graph 328 can be thought of as viewing the amount of compression occurring in the contraction graph 308 (e.g., the compression of the curtain or ribbon). The greater the curve, the higher the time scale, because a larger amount of time is represented by a smaller horizontal distance.

FIG. 3D illustrates an example of an alternative secondary graph 330 (e.g., a secondary presentation, a compression graph) used to illustrate the amount of longitudinal data/graph occurring across the compressed areas of the primary graph (e.g., the main longitudinal graph, the contraction graph 308, the primary presentation). The reference numbers used in FIGS. 3A and 3B have been reproduced in FIG. 3D to indicate the same or similar elements. In the example shown in FIG. 3D, the contraction graph 308 may be considered (e.g., thought of and/or analogized to) as a three-dimensional graph (e.g., a piece of graph paper) where the areas 318A-F (e.g., the compressed areas, the areas not showing contractions) are represented as curves (e.g., folds, creases) in the graph 308 that are bent inwards (e.g., into the screen 300, away from the viewer) to reduce the horizontal display area taken up by the areas 318A-F.

In the example shown in FIG. 3D, the line shown in secondary graph 330 represents the top edge of the three-dimensional graph and displays the curves and bends occurring in the graph 308. Relatively small bends or curves in the graph 308 occur at the areas 318A-D. These areas of the graph 308 appear to bend or curve into the screen and, thus, the points of interest (e.g., the contractions 316A-F) are moved closer together. In some examples, a compressed area may bent far enough where the graph 308 folds back onto itself (e.g., forms a bulge similar to a curtain contacting a wall and bowing outwards). The shape of the graph 308 occurring at compressed area 318E (e.g., the non-contraction area) can be seen in the secondary graph 330, and, as shown, the area 318E of the graph 308 bends backwards and then folds back onto itself (e.g., forms a bulge). The area 318F is shown in the secondary graph 308 as a sharper bend in the graph 308. The data occurring during the compressed area 318F can still be seen in the graph 308 because none of the graph 308 folds back on to itself or is obstructed by other sections of the graph 308.

The secondary graph 330 shown in FIG. 3D allows a viewer to see the amount of compression occurring across the compressed areas by displaying what the top of a 3D graph would look like having been bent or curved (e.g., an amount or degree of bulging) to compress uninteresting portions of the data and to move the points of interest closer together. The secondary graph 330 may be provided by, for example, the compression graph module 212 of the presentation system 102 shown in FIG. 2. The compression graph module 212 determines the visual appearance of bending the graph and presents these bends or compression as the secondary graph to indicate the amount of compression occurring in the primary graph.

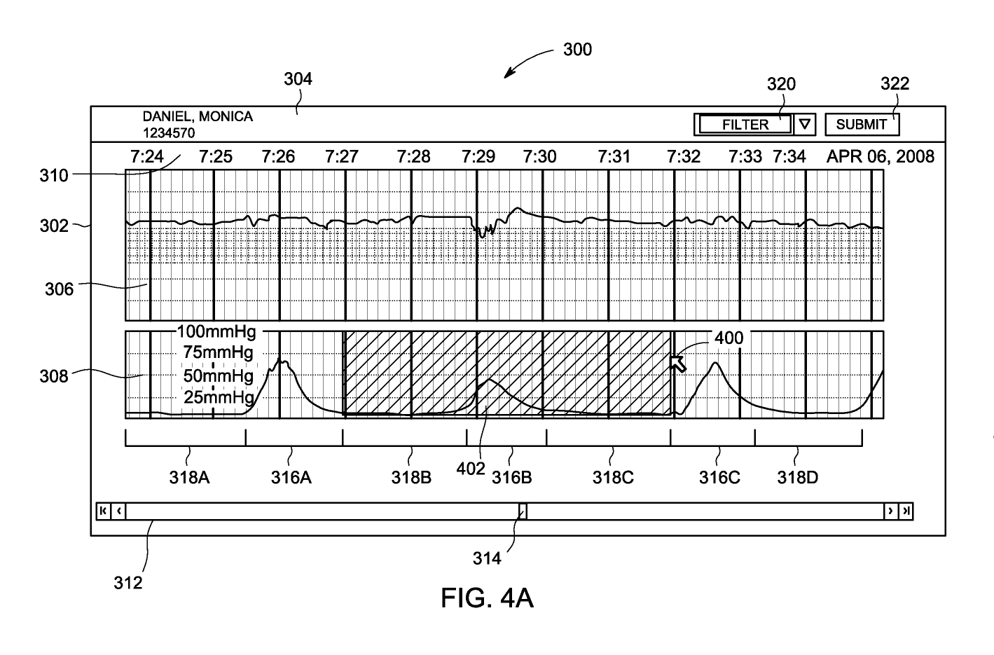

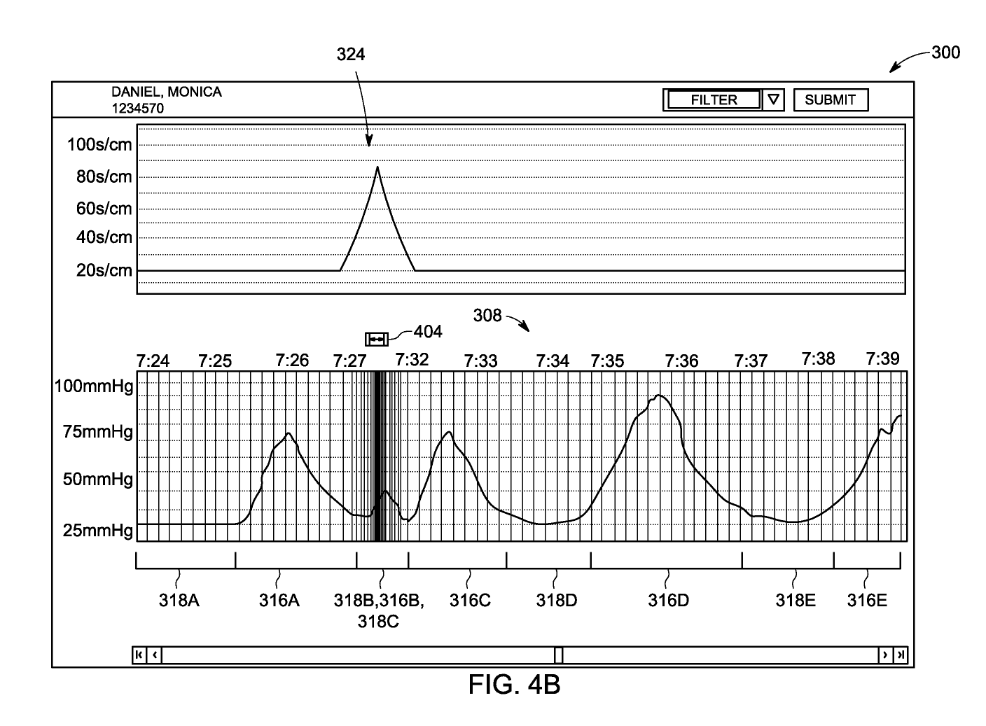

In some examples, instead of and/or in addition to automatically identifying the POIs or ranges of the data/graph that lack POIs, a user may select areas of the data/graph to compress or reduce in display size. FIG. 4A illustrates the example screen 300 and the application window 302 from FIG. 3A. The reference numbers used in FIG. 3A have been reproduced in FIG. 4A to indicate the same or similar elements. The upper graph 306 shows the fetal heat rate and the lower graph 308 shows the contractions of the mother. In some examples, a user may want to view only certain POIs (e.g., only certain types contractions or specific features in the signal). For example, a doctor may want to view only contractions above 50 mm/Hg. As shown in the lower graph 308, the data include contractions 316A, 316B, 316C and non-contraction areas 318A, 318B, 318C, 318D. Contraction 316B is under 50 mm/Hg. Therefore, a doctor may desire to view the contractions above 50 mm/Hg, i.e., contraction 316A and contraction 316C. In this example, the user may highlight or select a range (e.g., a section, a portion, an area, a segment) of the data/graph to reduce in display size (e.g., a range of data lacking POIs). As shown in FIG. 4A, an arrow 400 (e.g., controlled by a mouse) has been used to highlight a section 402 of the graph that the user wishes to reduce in display size. In this example, the highlighted section 402 includes the non-contraction area 418B, the contraction 416B and the non-contraction area 418C. In some examples, the highlighted section 402 may be displayed in a different shade or color over the graph 308. In some examples, after highlighting an area to be compressed, the user clicks the submit button 322 and the presentation system processes the request to reduce the section 402 selected by the user. In the example presentation system 102 shown in FIG. 2, the presentation system 102 includes a user selection module 210, which receives requests from the user to reduce a selected section of the graph.

FIG. 4B illustrates the example graph 308 of FIG. 4A after the three areas have been reduced. As shown, the non-contraction area 318B, the contraction 316B and the non-contraction area 318C have been compressed so that the contractions 316A, 316C are located closer together in the viewable display area. By compressing the three areas 318B, 316B, 318C, more of the longitudinal data/graph is shown in the application window 302. The user may continue to highlight and compress areas of the data/graph 308. In the example shown in FIGS. 4A and 4B, the section 402 of the data/graph was highlighted to compress the highlighted section 402. However, in other examples, the opposite can also be performed. In other words, in some examples, a user may highlight the sections of the data/graph the user wants to retain. After highlighting such areas, the presentation system compresses the remaining areas (e.g., the areas between the highlight areas) so that the POIs are displayed closer together.

Additionally or alternatively, in some examples, a user may select two points in the data/graph and move these points together on the screen 300 (e.g., by clicking and dragging one of the points towards the other point) to compress the data occurring between the two points. For example, a user may select a first point occurring at the beginning of the non-contraction area 318B and a second point occurring at the end of the non-contraction area 318C. Then, the user may click and drag the points closer together to compress the non-contractions areas 318B and 318C and the contraction 318B (e.g., resulting in the display shown in FIG. 4B). In this example, the user manually controls the amount of compression occurring between the two points.

Also shown in FIG. 4B is the secondary graph 324 that displays the time scale occurring in the graph 308. As shown, there is a spike in the time scale occurring where the user highlighted and compressed the unimportant data/graph. The time scale occurring during areas 318B, 316B, 318C increases and decreases. Similar to the curtain effect described above, the compressed area appears as a bend or curve in the graph, which brings the contractions 316A, 316C closer together (e.g., visual co-location).

As shown in FIG. 4B, an expand button 404 is displayed adjacent the compressed area. The expand button 404 indicates to a user that an area has been compressed and the user may select the expand button 404 in order to expand the compressed area (e.g., the non-contractions 318B, 318C and the contraction 316B).

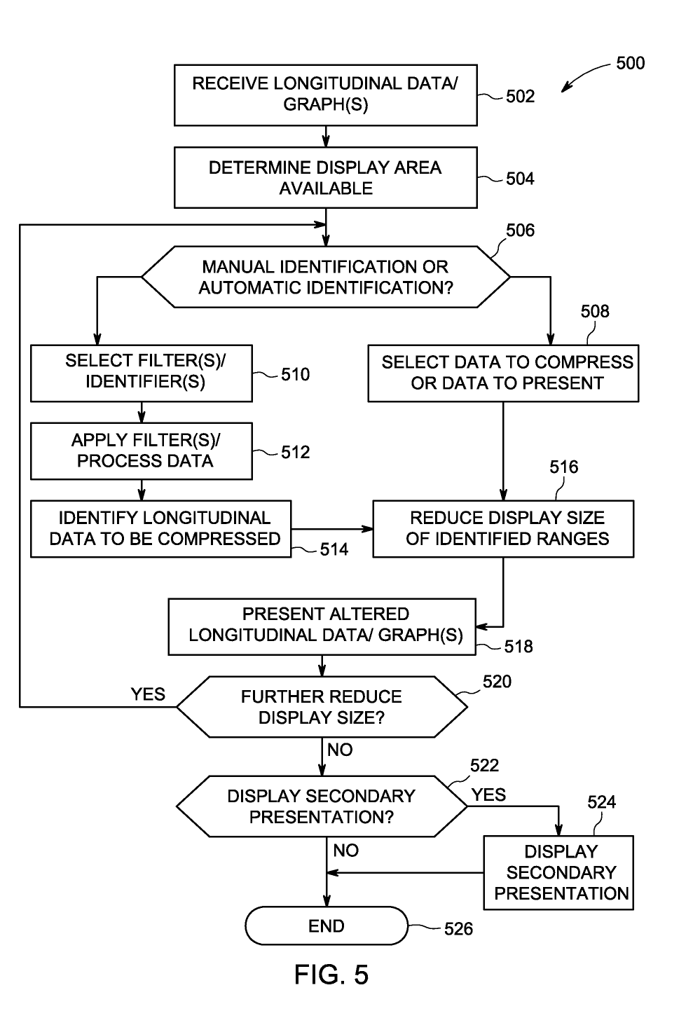

While an example manner of implementing the longitudinal data presentation system 102 of FIG. 2 and/or the presentation displays of FIGS. 3A-4B is illustrated in FIG. 5, one or more of the elements, processes and/or devices illustrated in FIG. 5 may be combined, divided, re-arranged, omitted, eliminated and/or implemented in any other way. Further, the example display module 200, the example input module 202, the example filter/identifier database 204, the example comparator 206, the example presentation adjuster 208, the example user selection module 210, the example compression graph module 212 and/or, more generally, the example longitudinal data presentation system 102 of FIG. 2 may be implemented by hardware, software, firmware and/or any combination of hardware, software and/or firmware. Thus, for example, any of the example display module 200, the example input module 202, the example filter/identifier database 204, the example comparator 206, the example presentation adjuster 208, the example user selection module 210, the example compression graph module 212 and/or, more generally, the example longitudinal data presentation system 102 could be implemented by one or more analog or digital circuit(s), logic circuits, programmable processor(s), application specific integrated circuit(s) (ASIC(s)), programmable logic device(s) (PLD(s)) and/or field programmable logic device(s) (FPLD(s)). When reading any of the apparatus or system claims of this patent to cover a purely software and/or firmware implementation, at least one of the example display module 200, the example input module 202, the example filter/identifier database 204, the example comparator 206, the example presentation adjuster 208, the example user selection module 210 and/or the example compression graph module 212 is/are hereby expressly defined to include a tangible computer readable storage device or storage disk such as a memory, a digital versatile disk (DVD), a compact disk (CD), a Blu-ray disk, etc. storing the software and/or firmware. Further still, the example longitudinal data presentation system 102 of FIG. 2 may include one or more elements, processes and/or devices in addition to, or instead of, those illustrated in FIG. 5, and/or may include more than one of any or all of the illustrated elements, processes and devices.

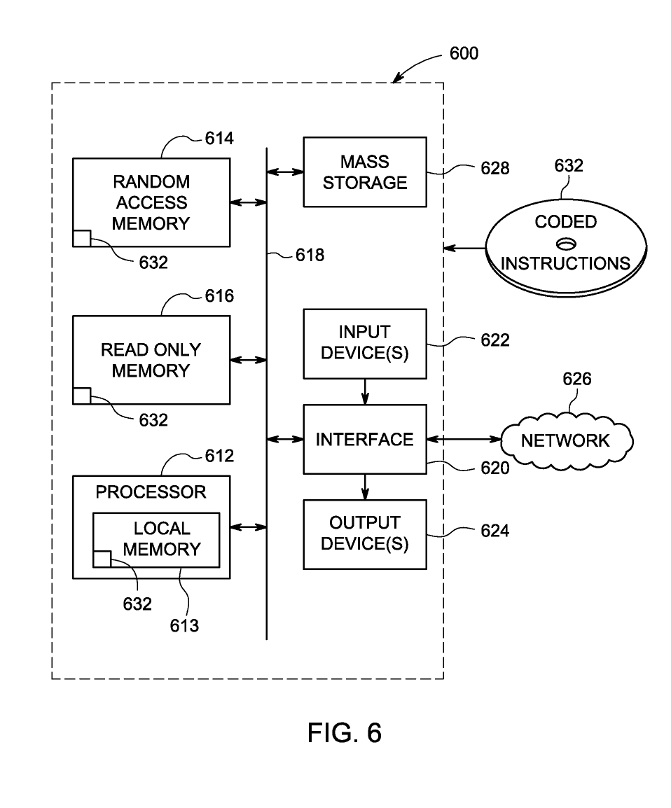

A flowchart representative of example machine readable instructions for implementing the example longitudinal data presentation system 102 of FIG. 2 and/or the example presentation displays of FIGS. 3A-4B is shown in FIG. 5. In this example, the machine readable instructions comprise a program for execution by a processor such as the processor 612 shown in the example processor platform 600 discussed below in connection with FIG. 6. The program may be embodied in software stored on a tangible computer readable storage medium such as a CD-ROM, a floppy disk, a hard drive, a digital versatile disk (DVD), a Blu-ray disk, or a memory associated with the processor 612, but the entire program and/or parts thereof could alternatively be executed by a device other than the processor 612 and/or embodied in firmware or dedicated hardware. Further, although the example program is described with reference to the flowchart illustrated in FIG. 5, many other methods of implementing the example longitudinal data presentation system 102 and/or the example presentation displays of FIGS. 3A-4B may alternatively be used. For example, the order of execution of the blocks may be changed, and/or some of the blocks described may be changed, eliminated, or combined.

As mentioned above, the example processes of FIG. 5 may be implemented using coded instructions (e.g., computer and/or machine readable instructions) stored on a tangible computer readable storage medium such as a hard disk drive, a flash memory, a read-only memory (ROM), a compact disk (CD), a digital versatile disk (DVD), a cache, a random-access memory (RAM) and/or any other storage device or storage disk in which information is stored for any duration (e.g., for extended time periods, permanently, for brief instances, for temporarily buffering, and/or for caching of the information). As used herein, the term tangible computer readable storage medium is expressly defined to include any type of computer readable storage device and/or storage disk and to exclude propagating signals and to exclude transmission media. As used herein, “tangible computer readable storage medium” and “tangible machine readable storage medium” are used interchangeably. Additionally or alternatively, the example processes of FIG. 5 may be implemented using coded instructions (e.g., computer and/or machine readable instructions) stored on a non-transitory computer and/or machine readable medium such as a hard disk drive, a flash memory, a read-only memory, a compact disk, a digital versatile disk, a cache, a random-access memory and/or any other storage device or storage disk in which information is stored for any duration (e.g., for extended time periods, permanently, for brief instances, for temporarily buffering, and/or for caching of the information). As used herein, the term non-transitory computer readable medium is expressly defined to include any type of computer readable storage device and/or storage disk and to exclude propagating signals and to exclude transmission media. As used herein, when the phrase “at least” is used as the transition term in a preamble of a claim, it is open-ended in the same manner as the term “comprising” is open ended.

FIG. 5 illustrates a flow diagram of an example method 500 to identify POIs in a longitudinal data/graph and/or areas of the longitudinal data/graph not containing POIs and reduce the display size of the areas of longitudinal data/graph not containing POIs, and implementing the system 100, the longitudinal data presentation system 102, and the example presentation displays 300, shown in FIGS. 1-4. The method of FIG. 5 begins at block 502 with receiving the longitudinal data/graph(s). The longitudinal data/graph(s) may include any type of data/graph(s) having one variable measured against another variable (e.g., time). Longitudinal data may include discrete measurements (e.g., a sequence of individual data points) and/or continuous measurements. Examples of longitudinal data/graph(s) commonly used in the healthcare industry include EKGs, ECGs, blood pressure monitors, fetal heat beats, contractions, etc. The longitudinal data/graph(s) may be received by and/or stored in an instrument having a screen or user interface for viewing the data. In some examples, such as the system 100 shown in FIG. 1, the longitudinal data/graph(s) may be provided by an outside system 104, 106 or 108 and longitudinal data/graph(s) may be viewed on the workstation 114.

In some examples, longitudinal data/graph(s) are relatively long and include a plurality of POIs and ranges (e.g., portions, sections, areas, segments, etc.) not containing points of interest. To view the longitudinal data/graph(s), a user scrolls through the data/graph(s) (e.g., on a screen or on paper) to identify the POIs. In some examples, a screen displaying the longitudinal data/graph(s) has a finite area where the data/graph(s) can be displayed (e.g., screen real estate, a viewable display area). Therefore, only a relatively small section of the data/graph(s) is viewable at one time. When viewed on a screen, a user scrolls through the data on the screen. When viewed in paper, a user must manually scroll through the strip containing the data/graph(s).

At block 504, the amount of available screen space is determined. In some examples, the amount of compression is based on the amount of available screen space and/or resolution of the longitudinal data/graph(s). In the example presentation system 102 shown in FIG. 2, the display module 200, which interacts with the display screen, determines the amount of available screen space and/or resolution of the longitudinal data/graph(s) to be displayed.

At block 506, either manual identification (e.g., selection) or automatic identification of ranges of the longitudinal data/graph(s) to be compressed occurs. For example, in the system 102 shown in FIG. 2, and display screens 300 shown in FIGS. 3A-4B, a user may select automatic identification of the POIs or the user may manually select areas of the longitudinal data/graph(s) to compress.

If manual selection of the longitudinal data/graph(s) to be compressed occurs, at block 508 a user selects one or more ranges (e.g., portions, sections, areas, segments, etc.) of the data/graph(s) to be reduced in display size. In some examples, there are ranges of the data/graph(s) occurring between POIs that are unimportant or uninteresting to the user, i.e., the user may only be concerned with certain features in the data/graph(s). The user may select (e.g., highlight via a mouse) these ranges occurring between the POIs. For example, in the presentation display screens 300 of FIGS. 4A and 4B, a user highlights the non-contraction areas 418A and 418B and the contraction 416B. This range of the data/graph(s) represents a portion of the data/graph(s) between two POIs (e.g., contractions 316A and 316B) that the user wants to reduce in display size in order to better view the POIs. In other examples, the user may select two points in the in the data/graph(s) and then pull the two points closer together, thus compressing the area of the data/graph occurring between the two points.

In some examples, instead of highlighting or selecting the ranges of the data/graph(s) lacking POIs, the user may select the ranges of the data/graph(s) occurring over the POIs (e.g., the portions of the data the user desires to view) at block 508. In such an example, the user selects one or more ranges of the data/graph(s) to present, so the ranges of the data/graph(s) left out (e.g., the ranges of the data/graph(s) lacking POIs) can be identified and reduced in display size.

Additionally or alternatively, if automatic compression of the longitudinal data/graph(s) is to occur, a user may select one or more filters or identifiers at block 510. In some examples, certain waveforms, patterns, shapes and/or features in the longitudinal data/graph(s) are indicative of certain events. These waveforms, patterns, shapes and/or features are typically industry specific, and when viewing the longitudinal data/graph(s), it is often the case that only certain waveforms, patterns, shapes and/or features are sought in the longitudinal data/graph(s). Many of these waveforms or patterns known. At block 510, a filter or identifier corresponding to a desired waveform may be chosen. The filter or identifier captures ranges in the data/graph(s) that have a substantially corresponding or matching waveform, pattern, shape and/or feature. In the example presentation system 102 shown in FIG. 2, the system 102 includes a filter/identifier database 204 that stores one or more predetermined waveforms. A user may select one of the waveforms and apply it to the longitudinal data. For example, in presentation display 300 shown in FIG. 3A, the dropdown menu 320 includes a list of the stored filters/identifiers that a user may choose from.

At block 512, the selected filter/identifier is applied to the longitudinal data/graph(s). The selected filter/identifier is compared to the data/graph(s) to identify waveforms, patterns, shapes and/or features occurring in the data that substantially correspond or match the selected filter/identifier. The filters/identifiers may include a list of parameters including, for example, minimum and maximum time lengths for a respect waveform, relative amplitudes and/or other data that defines the respective waveform. Based on the selected filter/identifier, one or more waveforms in the data/graph(s) can be identified as POIs (e.g., waveforms or features that match the desired or selected filter). For example, in the presentation system 102 of FIG. 2, the system 102 includes a comparator 206 to compare the selected filter/identifier to the data/graph(s).

At block 514, the ranges of data falling outside of or between the points of interest (identified at block 512) are identified as ranges of the data/graph(s) to be reduced in display size. For example, in the presentation display screen 300 shown in FIG. 3B, the non-contraction areas 318A-F are identified as ranges of the data/graph(s) falling outside of or between the contractions.

At block 516, the display size of the identified ranges of the data/graph(s) are compressed. If a user manually selects or highlights the ranges of the data/graph(s) to reduce or the data/graph(s) to present at block 508, the respective range of the data/graph(s) identified is reduced in display size (e.g., compressed, altered, distorted, changed). Additionally or alternatively, if a filter/identifier is used to automatically identify such ranges at blocks 510-514, the ranges of the data/graph(s) identified as lacking POIs is reduced in display size. To reduce the display size of the ranges of the data/graph(s) lacking POIs, the time scale occurring during these ranges may be increased. Therefore, the length of time represented by a unit of length is increased, and a longer time span occurs over a shorter distance. This process positions the POIs visually closer to each other, while still preserving and presenting the trends in the reduced data ranges. The adjustment may be implemented, for example, using the presentation adjuster 208 of the presentation system 102 shown in FIG. 2. Also, as shown in FIGS. 3B and 3C, the presentation system reduces the display size of the non-contraction areas 318A-F by increasing the time scale occurring during the non-contractions areas 318A-F, which allows more POIs to be displayed in the application window 302 of the screen 300 (e.g., the viewable display area). In the example shown in FIGS. 3B and 3C, the non-contraction areas 318A-F are compressed using a time scale that changes (e.g., increases and/or decreases, linearly, non-linearly, exponentially). However, in other examples, these areas may be compressed using a time scale that is constant throughout the respective area (and with a higher time scale than the POIs). For example, the contraction area 316A may be displayed in a first time scale (e.g., 20 s/cm), the contraction area 316B may be displayed in a second time scale (e.g., 20 s/cm), and the non-contraction area 318B may be displayed in a third time scale (e.g., 40 s/cm). In this example, the non-contraction area 318B occurs over a time scale that is double the time scale of the contraction areas 316A, 316B occurring on either side (e.g., occurring before and/or after) and, thus, the non-contraction area 318B takes up less display space in the application window 302 and brings the contraction areas 316A, 316B closer together.

In some examples, the amount of compression is based on the amount of display space available (e.g., determined at block 504) and/or the amount of the data/graph to be displayed. For example, if the longitudinal graph is relatively long, but only a few POIs are identified, the ranges of the data/graph between the POIs may be compressed to fit the POIs within the viewable display area.

At block 518, the altered longitudinal data/graph(s) is presented. At block 520, it is determined whether the longitudinal data/graph(s) is to be further altered (e.g., by applying another filter/identifier). If a user desires to further reduce the display size of certain ranges of the data/graph(s), the process begins again at block 506 where either manual or automatic identification can occur.

At block 522, it is determined whether a secondary presentation is to display the compression occurring in the longitudinal graph. A secondary presentation (e.g., a compression graph) may be presented above or below the longitudinal graph to indicate the amount of compression(s) occurring at the various locations in the longitudinal graph. The secondary presentation provides a visual indication of the amount of time compression, so that a user may understand amount of time that has been compressed. For example, the presentation system 102 shown in FIG. 2 includes the compression graph module 214 that determines the amount of compression occurring in the longitudinal data/graph and displays a graph (e.g., adjacent the longitudinal data/graph) to indicate this compression. FIGS. 3C, 3D and 4B illustrate examples having the secondary graphs 328 (e.g., compression graphs) that visually display the amount of compression occurring in the contraction graph 308. If a compression graph is to be displayed, at block 524 a compression graph is presented. Otherwise, the process ends at block 526.

The example presentation systems and methods advantageously reduce the display size of ranges of data/graph in a longitudinal graph that lack POIs and bring the POIs closer together so that more POIs are viewable in the same display area. The example presentation systems and methods also preserve the data occurring in the reduced areas (e.g., the data is not deleted or omitted), so that the trends of this data are still viewable. Further, the example presentation systems and methods increase workflow efficiency, optimize recognition of data and reduce the time required to review POIs in a longitudinal graph.

FIG. 6 is a block diagram of an example processor platform 1000 capable of executing the instructions of FIG. 5 to implement the longitudinal data presentation system 102 of FIG. 2. The processor platform 600 can be, for example, a server, a personal computer, a mobile device (e.g., a cell phone, a smart phone, a tablet such as an iPad™), a personal digital assistant (PDA), an Internet appliance, a set top box, or any other type of computing device.

The processor platform 600 of the illustrated example includes a processor 612. The processor 612 of the illustrated example is hardware. For example, the processor 612 can be implemented by one or more integrated circuits, logic circuits, microprocessors or controllers from any desired family or manufacturer.

The processor 612 of the illustrated example includes a local memory 613 (e.g., a cache). The processor 612 of the illustrated example is in communication with a main memory including a volatile memory 614 and a non-volatile memory 616 via a bus 618. The volatile memory 614 may be implemented by Synchronous Dynamic Random Access Memory (SDRAM), Dynamic Random Access Memory (DRAM), RAMBUS Dynamic Random Access Memory (RDRAM) and/or any other type of random access memory device. The non-volatile memory 616 may be implemented by flash memory and/or any other desired type of memory device. Access to the main memory 614, 616 is controlled by a memory controller.

The processor platform 600 of the illustrated example also includes an interface circuit 620. The interface circuit 620 may be implemented by any type of interface standard, such as an Ethernet interface, a universal serial bus (USB), and/or a PCI express interface.

In the illustrated example, one or more input devices 622 are connected to the interface circuit 620. The input device(s) 622 permit(s) a user to enter data and commands into the processor 612. The input device(s) can be implemented by, for example, an audio sensor, a microphone, a camera (still or video), a keyboard, a button, a mouse, a touchscreen, a track-pad, a trackball, isopoint and/or a voice recognition system.

One or more output devices 624 are also connected to the interface circuit 620 of the illustrated example. The output devices 624 can be implemented, for example, by display devices (e.g., a light emitting diode (LED), an organic light emitting diode (OLED), a liquid crystal display, a cathode ray tube display (CRT), a touchscreen, a tactile output device, a light emitting diode (LED), a printer and/or speakers). The interface circuit 620 of the illustrated example, thus, typically includes a graphics driver card, a graphics driver chip or a graphics driver processor.

The interface circuit 620 of the illustrated example also includes a communication device such as a transmitter, a receiver, a transceiver, a modem and/or network interface card to facilitate exchange of data with external machines (e.g., computing devices of any kind) via a network 626 (e.g., an Ethernet connection, a digital subscriber line (DSL), a telephone line, coaxial cable, a cellular telephone system, etc.).

The processor platform 600 of the illustrated example also includes one or more mass storage devices 628 for storing software and/or data. Examples of such mass storage devices 628 include floppy disk drives, hard drive disks, compact disk drives, Blu-ray disk drives, RAID systems, and digital versatile disk (DVD) drives.

The coded instructions 632 of FIG. 5 may be stored in the mass storage device 628, in the volatile memory 614, in the non-volatile memory 616, and/or on a removable tangible computer readable storage medium such as a CD or DVD.

Although certain example methods, systems and tangible machine readable storage mediums have been disclosed herein, the scope of coverage of this patent is not limited thereto. On the contrary, this patent covers all methods, systems and tangible machine readable storage medium fairly falling within the scope of the claims of this patent.

Additionally or alternatively, while the invention has been described with reference to certain embodiments, it will be understood by those skilled in the art that various changes may be made and equivalents may be substituted without departing from the scope of the invention. In addition, many modifications may be made to adapt a particular situation or material to the teachings of the invention without departing from its scope. Therefore, it is intended that the invention not be limited to the particular embodiment disclosed, but that the invention will include all embodiments falling within the scope of the appended claims.

What is claimed is:

1. A method for displaying longitudinal data, the method comprising:
receiving the longitudinal data comprising a plurality of measurements of a variable occurring over a period of time;
identifying a first point of interest and a second point of interest in the longitudinal data;
displaying, in a first graph, the first point of interest and the second point of interest in a first time scale; and
displaying, in the first graph, a portion of the longitudinal data occurring between the first point of interest and the second point of interest in a second time scale, the second time scale different than the first time scale.

2. The method of claim 1, wherein identifying the first point of interest and the second point of interest comprises comparing the longitudinal data with a selected waveform.

3. The method of claim 1, wherein the longitudinal data comprises fetal heart rate data.

4. The method of claim 1, wherein the first time scale is substantially constant throughout the first point of interest and the second point of interest.

5. The method of claim 1, wherein the second time scale is greater than the first time scale.

6. The method of claim 1, wherein the first point of interest and the second point of interest are identified by a user.

7. The method of claim 1, wherein the second time scale at least one of increases or decreases throughout the portion of the longitudinal data occurring between the first point of interest and the second point of interest.

8. The method of claim 1 further comprising displaying a second graph showing the time scale occurring in the first graph.

9. The method of claim 8, wherein the second graph is displayed vertically above or below the first graph.

10. A system to display longitudinal data, the system comprising:
an input module to receive longitudinal data, the longitudinal data comprising a variable measured over a period of time;
a processor to:
identify a first point of interest and a second point of interest in the longitudinal data; and
identify a segment of the longitudinal data occurring between the first point of interest and the second point of interest; and
a dashboard to display a primary graph showing the first point of interest in a first time scale, the second point of interest in a second time scale and the segment of the longitudinal data occurring between the first and second points of interest in a third time scale, the third time scale different than the first and second time scales.

11. The system of claim 10, wherein the time scale comprises an amount of time per unit of distance in the graph.

12. The system of claim 10, wherein at least one of the first time scale or the second time scale is substantially constant.

13. The system of claim 12, wherein the third time scale at least one of increases or decreases throughout the segment of the longitudinal data occurring between the first point of interest and the second point of interest.

14. The system of claim 13, wherein the third time scale at least one of increases or decreases exponentially.

15. The system of claim 10, wherein the dashboard is to display a secondary graph indicating the time scale occurring in the primary graph.

16. The system of claim 15, wherein the dashboard is to display the secondary graph above or below the primary graph.

17. A tangible machine readable storage medium comprising instructions that, when executed, cause a machine to at least:
identify a point of interest in longitudinal data;
identify a first section of the longitudinal data occurring prior to the point of interest and a second section of the longitudinal data occurring after the point of interest; and
display, via a graphical user interface, the longitudinal data in a longitudinal graph, wherein the first section of the longitudinal data is displayed in a first time scale, the second section of the longitudinal data is displayed in a second time scale and the point of interest is displayed in a third time scale, wherein the third time scale is different than the first time scale and the second time scale.

18. The tangible machine readable storage medium of claim 17, wherein at least one of the first time scale and the second time scale is a non-linear time scale.

19. The tangible machine readable storage medium of claim 17, wherein the instructions, when executed, further cause the machine to display, via the graphical user interface, a time scale graph showing the time scale of the longitudinal data occurring in the longitudinal graph.

20. The tangible machine readable storage medium of claim 17, wherein the first section of the longitudinal data and the second section of the longitudinal data do not include points of interest.
